(19) United States
(12) Patent Application Publication
(10) Pub. No.: US 2002/0143143 A1
(43) Pub. Date: Oct. 3, 2002

(54) Peptides designed for the diagnosis and treatment of rheumatoid arthritis

(76) Inventors: Ann Union, Aalter (BE); Henri Moereels, Ekeren (BE); Lydie Meheus, Merelbeke (BE)

Correspondence Address: Patricia A. Kammerer, Howrey Simon Arnold & White, LLP, 750 Bering Drive, Houston, TX, 77057-2198, US

(21) Appl. No.: 09/747,029

(22) Filed: Dec. 21, 2000

(30) Foreign Application Priority Data
Dec. 21, 1999 (EP) 99870280.7
Sep. 8, 2000 (EP) 00870195.5

(51) Int. Cl. G01N 33/53; C07K 7/64

(52) U.S. Cl. 530/326; 530/350; 435/7.1; 435/7.5

(57) ABSTRACT

The present invention relates to peptides that mimic the immunogenic determinants of self-proteins recognized by autoimmune antibodies in a biological sample from patients suffering from rheumatoid arthritis (RA). More particularly, the present invention relates to citrulline-containing peptides, which react with the majority of the latter antibodies. Furthermore, the present invention relates to diagnostic tools for a more convenient and sensitive diagnosis of RA and to therapeutic methods to treat RA.

### FIELD OF THE INVENTION

The present invention relates to peptides that mimic the immunogenic determinants of self-proteins recognised by autoimmune antibodies in a biological sample from patients suffering from rheumatoid arthritis (RA). More particularly, the present invention relates to citrulline-containing peptides, which react with the majority of the latter antibodies. Furthermore, the present invention relates to diagnostic tools for a more convenient and sensitive diagnosis of RA and to therapeutic methods to treat RA.

### BACKGROUND OF THE INVENTION

Rheumatoid arthritis is a major crippling joint disease, which is systemic in nature and of unknown etiology. It affects 1% of the population, with a male to female ratio of 2:3. In terms of morbidity, the most important feature of RA is joint erosion which leads to pain, deformity and in some cases, severe disability. Life expectations in patients with a severe form of the disease are reduced by up to 10 years. RA has all the features of an autoimmune disease, including the presence of a variety of autoantibodies in patients' sera and the capacity to induce illness by transfer of pathogenic T cells in animal models. The classification of the disease can be challenged on the grounds that borderline forms are very common; furthermore inflammation of the joints is not only restricted to RA, but occurs also in other non-autoimmune diseases such as osteoarthritis, reactive arthritis and gout.

The diagnosis of rheumatoid arthritis is initially based on clinical manifestations. As an early diagnosis allows an adjusted treatment, which can highly improve life quality of the patients, it is of major importance for rheumatologists to have reliable diagnostic criteria at their disposal. Serological support for diagnosing RA is not very well established and is based mainly on the presence of rheumatoid factors (RF). However, a substantial number of RA patients are RF-negative, while on the other hand, RF is also present in other rheumatic diseases including Sjogren's syndrome and systemic lupus erythematosus, in some chronic bacterial and acute viral infections, in certain parasitic diseases and chronic inflammatory diseases, and has furthermore been demonstrated in sera from healthy persons. The rather low specificity of RF thus necessitates additional testing for a second RA-specific antibody.

During recent years, much effort has been put in the identification of novel RA-specific autoantigens that are usable in a diagnostic test kit in order to facilitate early and specific diagnosis. Until now however, no commercial product has been entering the market yet. In this respect, Vincent et al. (1999) demonstrated the usefulness of natural filaggrin on Western blot. At a specificity level of 98.6% using 213 disease control sera, 159/279 (37%) RA sera showed positive reaction on Western blot. Natural filaggrin was also applied onto ELISA plates, which could detect 47% of 55 RA sera, while only 1% of healthy controls showed positivity (Palosuo et al., 1998). However, filaggrin is isolated from human epidermis, which is not convenient for usage in a diagnostic kit. Also the APF (antiperinuclear factor) test, which is highly sensitive for rheumatoid arthritis, is generally not used as diagnostic test for rheumatoid arthritis because of the subjectivity and laboriousness of the immunofluorescence technique, the necessity to use preselected buccal cell donors, the problematical interlaboratory standardisation and the rather low specificity.

The modified Arg residue citrulline (Cit) was shown to constitute an indispensable modification necessary to obtain proper immune recognition of filaggrin by the autoantibodies present in human RA sera (Girbal-Neuhauser et al., 1999). In vitro deimination of recombinant human filaggrin by the enzyme peptidylarginine deiminase (PAD) generated a protein reactive with human RA sera in ELISA, while the non-citrullinated protein proved unreactive. This was further confirmed, by testing the reactivity of three citrulline-substituted filaggrin-derived synthetic peptides called E12Dcit, T12Ecit and El2Hcit in comparison with the unsubstituted related peptides E12D, T12E and E12H. Two of the three citrulline-substituted synthetic peptides, E12Dcit and E12Hcit, were recognised in ELISA by a small series of affinity-purified anti-filaggrin autoantibodies, whereas the unsubstituted related peptides were unreactive (Girbal-Neuhauser et al., 1999). Other citrulline-containing filaggrin-related peptides were also synthesised by Schellekens et al. (1998) and evaluated in ELISA. Selection of the peptides was deduced from filaggrin sequences containing a large number of Arg residues, or from regions with high antigenicity index or high turn probability. RA sera showed different patterns of reactivity with these peptide variants. The highest sensitivity obtained with an individual peptide was 48% in a group of 134 RA sera at a specificity of >98% against disease control and healthy subjects.

The patent application WO 99/28344 furthermore revealed that such citrulline-containing epitopes could be demonstrated in natural filaggrin and indeed were immunoreactive in a line immunoassay (LIA) system with ±50% of a series of 107 RA sera at a specificity level of 95% against healthy and disease controls.

In the present invention citrulline-containing peptides were generated which surprisingly demonstrate a more sensitive immunoreactivity with RA autoantibodies then filaggrin-derived citrulline-substituted peptides described in the prior art.

Since RA therapy includes the administration of very aggressive drugs, therapy is only started when the patients clearly have RA symptoms and there is no doubt about the diagnosis. In most cases therapy is started when most damage has occurred. Boers et al. (1997) demonstrated that an early start of RA therapy with a combination of disease modifying drugs is very successful in preventing disease progression. Therefore, convenient diagnostic tools providing an early RA diagnosis with a high PPV (Positive Predictive Value) would be of great value. A diagnostic test with a high PPV refers to tests in which a positive result indicates with high certainty that the patient will develop RA. Therefore, there is a need for diagnostic tools which makes a very sensitive diagnosis of RA at a high specificity level possible.

### SUMMARY OF THE INVENTION

The citrulline-containing peptides of the present invention proved to be useful for developing a convenient and specific diagnostic tool making a sensitive diagnosis of RA possible. Using the citrullinated peptides of the present invention it is possible to develop a diagnostic tool for RA with a high specificity and a high PPV which also proved to be complementary with other diagnostic tools described in the art, like the determination of rheumatoid factors (RF) in biological samples of RA patients . The high PPV together with the complementaity with other serological test, like RF determination, makes the retrieval of anti-filaggrin antibodies in biological samples from RA patients, a valuable tool in the diagnosis of RA.

According to its main embodiment the present invention relates to peptides comprising a sequence of less than 50 amino acids characterised in that said peptides contain a peptide turn comprising at least one citrulline residue, less than 12 amino acids between two cysteine residues, with said citrulline residue being one of the amino acids between said cysteine residues and said peptide being specifically recognised by autoimmune antibodies from patients suffering from rheumatoid arthritis.

The aim of the present invention is to provide citrulline-containing peptides that mimic the immunogenic determinants of self-proteins recognised by the immune system in patients suffering from rheumatoid arthritis and have a sensitive immunoreactivity with RA autoantibodies.

Another aim of the present invention is to provide methods for obtaining said peptides.

Another aim of the present invention is to provide methods of raising antibodies specifically reactive with said peptides.

Another aim of the present invention is to provide methods of raising anti-idiotype antibodies specifically reactive with the aforementioned antibodies, thereby mimicking said peptides.

Another aim of the present invention is to provide a diagnostic kit, which is highly specific for rheumatoid arthritis diagnosis.

Another aim of the present invention is to provide a pharmaceutical composition comprising said peptides, for therapy or diagnosis.

All these aims of the present invention are met by the following embodiments of the present invention.

### BRIEF DESCRIPTION OF THE FIGURES

FIG. 1: Three-dimensional structure of peptide IGP 1651. The three-dimensional structure can be described as follows: A: peptide strand-1, B: a turn of four residues and C: peptide strand-2. Each amino acid is determined by its position (1-18).

FIG. 2: Reactivity of purified synthetic peptides (IGP1611, IGP1646, IGP1650, IGP1651, IGP1684, IGP1685, IGP1686, IGP1687) with 48 RA sera on LIA. Specificity level >95%.

FIG. 3: Reactivity on LIA of the cyclic and linear forms of four synthetic peptides (a: IGP1611, b: IGP1646, c: IGP1650, d: IGP1651).

FIG. 4: Comparison between LIA using synthetic peptides of the present invention (=Fg LIA), Western Blot with natural filaggrin (=Fg Blot) and the APF fluorescence test using human buccal cells (APF test) for the detection of RA specific autoantibodies. 412 human sera are tested: 153 disease controls (1), 47 sera from patients with early disease (less than 12 months of symptoms) (2) and 212 longstanding RA) (2) and 212 longstanding RA sera (more than 4 years of symptoms) (3).

FIG. 5: Positive predictive value of LIA3 and LIA2. An estimated prevalence of RA patients in the daily rheumatology clinic of 20% brings the PPV for LIA3 to 87.3% and for LIA2 to 90.7%

### DETAILED DESCRIPTION OF THE INVENTION

According to its main embodiment the present invention relates to peptides comprising a sequence of less than 50 amino acids characterised in that said peptides contain a peptide turn comprising at least one citrulline residue, less than 12 amino acids between two cysteine residues, with said citrulline residue being one of the amino acids between said cysteine residues and said peptide being specifically recognised by autoimmune antibodies from patients suffering from rheumatoid arthritis.

Whenever the expression ‘peptide containing less than 50 amino acids’ is used, this should be interpreted in a broad sense, as a means of circumscribing an essentially truncated version of the entire immunoreactive protein that still comprises the highly reactive domain characterised by the presence of citrulline residues. These peptides have a length of preferably 40, 30, 25, 20 or less amino acids.

Whenever the expressions ‘peptides specifically recognised by RA autoantibodies’ or ‘peptides specifically reactive with RA autoantibodies’ are used, it refers to peptides, which are mainly recognised by RA autoantibodies present in a biological sample from RA patients.

The term ‘biological sample’ intends a fluid or tissue of a mammalian individual (e.g. a human) that commonly contains antibodies produced by the individual, more particularly RA antibodies. Such components are well known in the art and include, without limitation, blood, plasma, serum, urine, spinal fluid, lymph fluid and synovial fluid.

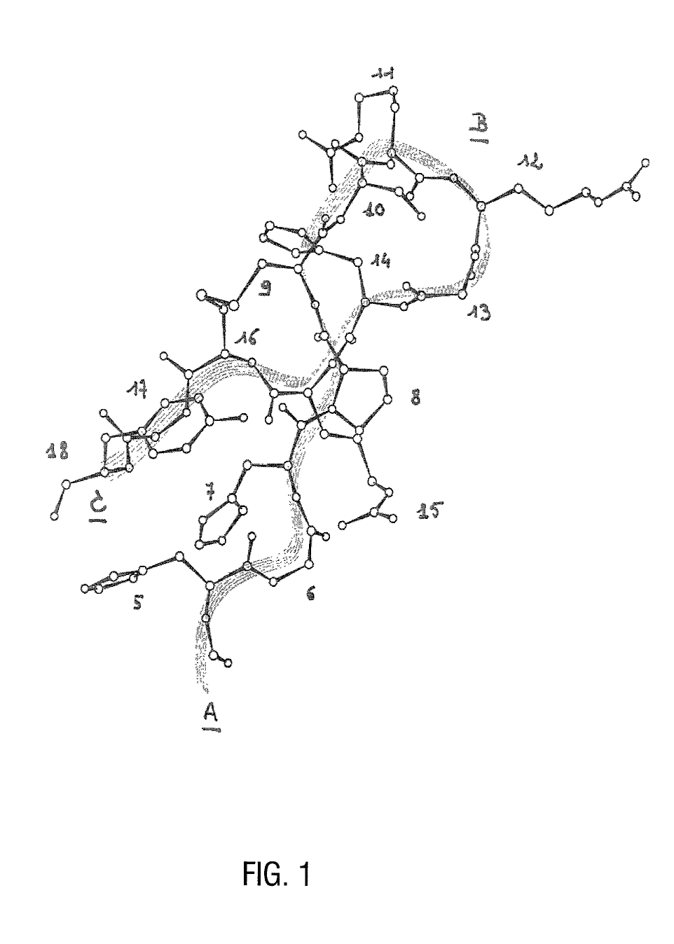

The term ‘peptide turn’ refers to any type of turn as observed experimentally in peptides and proteins. Among those turns some are classified as well known P-turns or y-turns. The turn can also obey a type with a specific description or be of an undefined type. Said ‘peptide turn’ can contain 1, 2, 3, 4 or even more amino acid residues. The peptide turn is further characterised by the critical presence of a citrulline residue, which plays a major role in the recognition process. Most preferably, the citrulline residue is located on top of the peptide turn described above (see FIG. 1, amino acid 12 refers to citrulline).

The present invention further relates to peptides described above, further characterised by the fact that said peptides are cyclic peptides.

The term cyclic peptide refers to any circularized form of said peptides.

According to a more specific embodiment the present invention relates to peptides described above circularized by any method known in the art, for example by the formation of a disulphide bound between the two cysteine residues.

The term ‘peptide’ as used throughout the specification and claims refers to a polymer of amino acids and does not refer to a specific length of the product; thus, oligopeptides, polypeptides and proteins are included within the definition of ‘peptide’. This term also does not exclude post-expression modifications of the peptide, for example, glycosylations, acetylations, phosphorylations and the like. Included within the definition are, for example, peptides containing one or more analogues or mimics of an amino acid (including, for example, unnatural amino acids, PNA, etc.), polypeptides with substituted linkages, as well as other modifications known in the art (including, for example amino acid replacement blocks obtained by organic chemistry), both naturally occurring and non-naturally occurring.

In a more specific embodiment the present invention relates to peptides described above which are biotinylated.

Chemical groups such as biotin may be added to either the amino- or carboxyl terminus creating a “linker arm” by which said peptides can conveniently be attached to a carrier. Other chemical groups such as, for example, thioglycolic acid, may be added to the termini which will endow said peptides with desired chemical or physical properties. Said “linker arm” can also be created by additional amino acids. In that case the “linker arm” will be at least one amino acid and may be as many as 60 amino acids but will most frequently be 1 to 10 amino acids. Natural amino acids such as histidine, cysteine, lysine, tyrosine, glutamic acid, or aspartic acid may be added to either the amino- or carboxyl terminus to provide functional groups for coupling to a solid phase or a carrier. The nature of the attachment to a solid phase or carrier can be non-covalent as well as covalent. Possible arrangements of this nature are well described in the art. N-terminal acetylation or terminal carboxy-amidation may also modify the termini of the said peptides, for example. In each instance, the peptide is as small as possible while still maintaining substantially all of the sensitivity of the larger peptide.

Biotinylated peptides can be obtained by any method known in the art, such as the one described in WO 93/18054 to De Leys.

The present invention further relates to peptides described above characterised in that said peptides are synthetic peptides.

The peptides of the present invention can be synthesised chemically or synthesised using well-known recombinant DNA techniques.

The present invention further relates to peptides described above characterised in that they contain 1, 2, 3, 4, 5, 6, 7, 8, 9, 10 or 11 residues between the two cysteine residues, with said citrulline residue being one of the amino acids between said cysteine residues.

In a more preferred embodiment the present invention relates to peptides described above characterised in that they contain 4 or 6 residues between the two cysteine residues, with said citrulline residue being one of the amino acids between said cysteine residues.

According to a further embodiment the present invention relates to peptides described above characterised in that said peptides have a length between 13 and 19 amino acids.

According to a more preferred embodiment the present invention relates to peptides described above characterised in that said peptides has a sequence containing 14, is 15, 16, 17 or 18 amino acids.

In a more specific embodiment the present invention relates to peptides described above characterised in that they have one of the following primary amino acid structures:

8 AA—Cysteine—2 AA—Citrulline—3 AA—Cysteine—2 AA or

5 AA—Cysteine—2 AA—Citrulline—3 AA—Cysteine—2 AA or

4 AA—Cysteine—2 AA—Citrulline—3 AA—Cysteine—2 AA or

8 AA—Cysteine—2 AA—Citrulline—1 AA—Cysteine—4 AA or

6 AA—Cysteine—2 AA—Citrulline—1 AA—Cysteine—4 AA or

4 AA—Cysteine—2 AA—Citrulline—1 AA—Cysteine—4 AA.

The abbreviation AA stands for any amino acid residue. The two cysteine residues in each peptide can form a disulphide bond.

The terms “amino acid”, “amino acid residue” or “residue” refers to all naturally occurring L-α-amino acids. The amino acids are identified by either the single-letter or three-letter nomenclature:

| | | | | | |
|---|---|---|---|---|---|
| Asp | D | aspartic acid | Ile | I | isoleucine |
| Thr | T | threonine | Leu | L | leucine |
| Ser | S | serine | Tyr | Y | tyrosine |
| Glu | E | glutamic acid | Phe | F | phenylalanine |
| Pro | P | proline | His | H | histidine |
| Gly | G | glycine | Lys | K | lysine |
| Ala | A | alanine | Arg | R | arginine |
| Cys | C | cysteine | Trp | W | tryptophan |
| Val | V | valine | Gln | Q | glutamine |
| Met | M | methionine | Asn | N | asparagine |

According to their primary amino acid structure “type I peptides”, as used in the present invention, are defined as those peptides of the present invention having 6 amino acid residues between the two cysteine residues. “Type II peptides”, as used in the present invention, are defined as those peptides of the present invention having 4 amino acids between the two cysteine residues.

The present invention also relates to peptides described above characterised in that the amino acids flanking the citrulline residue have a small volume and that they do not interact with the citrulline side chain providing a maximum accessibility of the citrulline residue.

More particularly, the present invention relates to peptides described above comprising one of the amino acid sequences presented in table 1.

All of the peptides described above were designed by molecular modelling and computational chemistry using the software from MSI (Molecular Simulations in Parc Club Orsay université, 20 rue J. Rostand, F-91893 Orsay Cedex, France). We used InsightII (97.2) for the modelling and Discover for the computational chemistry. Based on three-dimensional co-ordinates any molecule can be considered in the method of molecular modelling. The molecules raise from organic compounds, lipids, peptides to proteins. The co-ordinates can come from experiments like for example X-ray or NMR (Nuclear Magnetic Resonance) or are provided by molecular models obtained by building the molecule. Any type of molecule can be build in an atom wise way, which means atom per atom or by using a library containing a large number of fragments, for instance a phenyl ring or amino acids.

TABLE 1
Particular peptides of the present invention. ‘X’ refers to a citrulline residue.
Each amino acid residue can be defined by its position.

| Position | 1 | 2 | 3 | 4 | 5 | 6 | 7 | 8 | 9 | 10 | 11 | 12 | 13 | 14 | 15 | 16 | 17 | 18 |
|---|---|---|---|---|---|---|---|---|---|---|---|---|---|---|---|---|---|---|
| IGP1611 | Q | D | T | I | H | G | H | P | C | S | X | X | G | H | R | C | G | Y |
| IGP1646 | Q | D | T | I | H | G | H | P | C | S | S | X | G | H | R | C | G | Y |
| IGP1647 | Q | D | T | I | H | G | H | P | C | S | X | X | G | H | Q | C | G | Y |
| IGP1648 | Q | D | T | I | H | G | H | P | C | S | X | X | G | H | R | C | G | Q |
| IGP1649 | Q | D | T | I | H | G | H | P | C | S | X | X | G | H | Q | C | G | Q |
| IGP1650 | Q | D | T | I | H | G | H | P | C | S | X | X | G | C | R | P | G | Y |
| IGP1651 | | | | | H | G | H | P | C | S | X | X | G | H | R | C | G | Y |
| IGP1676 | | | | | H | G | H | P | C | S | X | X | G | C | R | P | G | Y |
| IGP1687 | | | | | H | G | H | G | C | D | X | X | G | H | R | C | G | Q |
| IGP1684 | | | | | H | G | H | G | C | D | S | X | G | H | R | C | G | Q |
| IGP1685 | Q | D | T | I | V | G | W | G | C | D | S | X | G | C | R | P | G | Q |
| IGP1686 | | | | | V | G | W | G | C | D | S | X | G | C | R | P | G | Q |

In the building procedure the user governs the three-dimensional structure of the molecule. This means that any conformation can be introduced, even unstable conformations. In fact the conformation is energy dependent and is also influenced by the molecular environment, temperature, charges etc. In consensus, the conformation is dependent on a large number of physico-chemical parameters. By using the method of computational chemistry the stability of the model can be calculated. In order to do so it calculates the energy of the molecule. According to the users skill, it can result in finding the lowest energy conformation of the molecule. For this lowest energy search, the conjugate gradient algorithm with the force field, which is called cvff, is used. Since the molecular structure moves, the founded lowest energy conformation is however a structure in a frozen status. By considering the structure, in fact the movements of the structure, in a period of time we can evaluate how robust the structure is or eventually find new energy minima. For this analysis we used molecular dynamics in a time period of 200000 pico seconds. The final molecular conformation is the result of an interplay between energy minimizations and molecular dynamics.

In the present invention many thousands of peptide conformations were considered and analysed by changing the backbone and the amino acid side chain conformations. Molecular modelling and computational chemistry offers the possibility IS of a directed approach in contrast to at random synthesis or combinatorial chemistry techniques. Using the described methods the present invention revealed that all peptides which are reactive with the autoimmune antibodies have a similar three-dimensional structure which can be described as follows: peptide strand-1, a turn of four residues, peptide strand-2. The citrulline residue is located on top of the turn. Each strand contains a cysteine residue. Both cysteine residues form a disulphide bond. (see FIG. 1). In general the turn contains four residues. However depending on the found lowest energy minimum conformation the turn can also contain three, two or one residue.

An essential part in the design of the resulting peptides is to evaluate each position in the peptide for confirmation with the three-dimensional structure by introducing different residues. Some residues in a particular position will result in minor modifications of the three-dimensional structure while other residues will introduce a new three-dimensional structure. The more the structure of the designed peptides deviates from the three-dimensional structure, the more the immunoreactivity with the rheumatoid arthritis sera declines.

Analysis of the three-dimensional structure of the peptides described in table 1 revealed that some side chains made specific interactions. For those peptides each amino acid residue is defined by its position. Surprisingly some residue interactions appear to be essential in the formation of the three-dimensional structure and consequently are essential for immunoreaction of said peptides with said autoimmune sera. The serine residue at position 10 (see table 1) before the first citrulline was observed to be involved in a hydrogen bond with the backbone of the turn or with the arginine side chain (position 15). As such it was mutated to an aspartic acid that also was able to form a like hydrogen bond. This peptide is also immunoreactive with autoantibodies present in sera from patients suffering from rheumatoid arthritis. Other interactions essential in the formation of the three-dimensional structure occurs between positions 5 and 7 and between positions 7 and 15 (see table 1).

The present invention also relates to other peptides, which can be generated by introducing other residues, which have a character for interactions. These interactions can be formed for example between basic and acidic residues or between aromatic residues. On the other hand these aromatic residues can also be combined in an interaction with basic or acidic residues. By the analysis of conformations close to the selected energy minimum conformation it was demonstrated in the present invention that the first citrulline residue made contact with other residues in the peptide. The main consequence is that this citrulline residue has a very low accessibility and can be changed to residues with a small non disturbing small volume (for example glycine or serine) leading to peptides that are immunoreactive with autoantibodies present in sera from patients suffering from rheumatoid arthritis.

Further analysis of the different peptide structures described above revealed additional specific interactions between residues, which are a prerequisite for immunoreaction of the designed peptides with autoantibodies present in sera from patients suffering from rheumatoid arthritis. This can be described as follows:

a) Type I peptides: Cys—six residues—Cys:

8 AA—Cysteine—2 AA—Citrulline—3 AA—Cysteine—2 AA or

5 AA—Cysteine—2 AA—Citrulline—3 AA—Cysteine—2 AA or

4 AA—Cysteine—2 AA—Citrulline—3 AA—Cysteine—2 AA

Some examples of type I peptides are presented in table 1 (IGP 1611, 1646, 1647, 1648, 1649, 1651, 1687 and 1684).

Additional side chain interactions are observed between the following residues:

position 4 and position 5,

position 5 and position 18,

position 11 and position 14,

position 18 and the S-atom of the two cysteine residues at position 9 and 16.

The present invention further demonstrates that the side chain of the isoleucine is residue (position 4) in the designed peptide, is not in favour for hydrogen bonding interaction with other residues. This residue is considered as being neutral and performs a border for polar interactions. After this residue polar interactions are possible between the residues located in the strands as stated above. The residues before this isoleucine residue do not influence the three-dimensional structure. These residues are forming a new unit that does not interact with the residues of strand-1, strand-2 or the turn residues. The only condition for these residues is that they can form a self-interacting unit. This means that within this unit each residue can find another unit residue that satisfies its own search for interaction. A triplet of residues with polar side chains accomplishes this feature. The same holds for a triplet with aliphatic and/or aromatic residues. Taking the above observations into account, peptides lacking the first three positions or lacking the first four positions still obey the three-dimensional structure and still are immunoreactive with autoantibodies present in sera from RA patients. The same observation holds for peptides with more than 6 residues between the two cysteine residues.

As a consequence the present invention relates to type I peptides described above characterised by said three-dimensional structure and the following criteria essential for accomplishing said structure and immunoreactivity with autoantibodies present in sera from rheumatoid arthritis patients:

1. Polar residue triplet preceding position 4.

2. A triplet of aliphatic and/or aromatic residues preceding position 4.

3. Turn residues 10, 11, 12, 13.

4. Residues 4, 5, 7, 9, 11, 14, 15, 16 and 18 that presents side chain interactions.

5. Peptides starting at position 4.

6. Peptides starting at position 5.

Table 2 presents all possible amino acids substitutions, which can be performed resulting in type I peptides of the present invention and meeting the three-dimensional structure, and side chain interactions described above which are essential for immunoreaction with autoantibodies present in body fluids from patients suffering from rheumatoid arthritis.

TABLE 2
Possible amino acid substitutions for type I peptides.

| Position | amino acid residue |
|---|---|
| 1 | Ser, Thr, Asp, Glu, Asn, Gln, His, Lys, Arg |
| 2 | Ser, Thr, Asp, Glu, Asn, Gln, His, Lys, Arg |
| 3 | Ser, Thr, Asp, Glu, Asn, Gln, His, Lys, Arg |
| 4 | Ile, Leu, Val, Ala, Met |
| 5 | His, Lys, Arg, Asp, Glu, Ser, Thr, Tyr, Phe, Trp |
| 6 | Gly |
| 7 | His, Lys, Arg, Asp, Glu, Ser, Thr, Tyr, Phe, Trp |
| 8 | Pro, Gly |
| 9 | Cys |
| 10 | Ser, Thr, Asp, Gly |
| 11 | Cit, Gly, Ser, Thr |
| 12 | Cit |
| 13 | Gly |
| 14 | His, Lys, Arg, Asp, Glu, Ser, Thr, Tyr, Phe, Trp |
| 15 | Arg, Lys, His, Asp, Glu, Ser, Thr, Gln, Asn, Tyr, Phe, Trp |
| 16 | Cys |
| 17 | Gly |
| 18 | Tyr, His, Lys, Arg, Asp, Glu, Ser, Thr, Gln, Asn, Phe, Trp |

Aliphatic and/or aromatic residues like alanine, valine, leucine, isoleucine, methionine, phenylalanine, tyrosine or tryptophan can also replace the amino acids indicated in position 1, 2 and 3.

The present invention also relates to shortened versions of the type I peptides described above in which the amino acids at position 1, 2 and 3 or at position 1, 2, 3 and 4 are omitted (for example IGP1651 in table 1).

b) Type II peptides: Cys—four residues—Cys peptides:

8 AA—Cysteine—2 AA—Citrulline—1 AA—Cysteine—4 AA or

6AA—Cysteine—2AA—Citrulline—AA-Cysteine—4AAor4AA—

Cysteine—2 AA—Citrulline—1 AA—Cysteine—4 AA.

Some examples of this type of peptides are presented in table 1 (IGP 1650, 1676, 1685 and 1686).

An additional side chain interaction is observed between the residue at position 4 and the residue at position 18 (see table 1). The present invention also revealed the importance of the length of the type II peptides as it governs an interaction pattern for the last residue of the peptide (for example Tyr). A type II peptide lacking the first four residues (position 1-4) was not recognised by the autoimmune antibodies of RA patients. A molecular dynamics study of these peptides showed that the three-dimensional structure as described above was no longer present. The reason for this feature is that the last residue (for example Tyr) has no valuable partner for interaction. Introduction at position 3 of an interaction partner for the last residue, for example Thr as interaction partner for Tyr, followed by a neutral border residue like isoleucine in position 4, re-establish the three-dimensional structure as described above and also the immunoreactivity with autoimmune antibodies from RA patients.

As a consequence the present invention relates to type II peptides described above characterised by said three-dimensional structure and the following criteria essential for accomplishing said structure and immunoreactivity with autoantibodies present in sera from rheumatoid arthritis patients:

Turn residues 10, 11, 12, 13

Residues 4, 5, 7, 15 and 18 that present side chain interactions

Peptides starting at position 3

Table 3 presents all possible amino acids substitutions which can be performed resulting in type II peptides of the present invention and meeting the three-dimensional structure and side chain interactions described above which are essential for immunoreaction with autoantibodies present in body fluids from patients suffering from rheumatoid arthritis.

TABLE 3
Possible amino acid substitutions for type II peptides.

| Position | amino acid residue |
|---|---|
| 1 | Ser, Thr, Asp, Glu, Asn, Gln, His, Lys, Arg |
| 2 | Ser, Thr, Asp, Glu, Asn, Gln, His, Lys, Arg |
| 3 | Ser, Thr, Asp, Glu, Asn, Gln, His, Lys, Arg |
| 4 | Ile, Leu, Val, Ala, Met |
| 5 | His, Lys, Arg, Asp, Glu, Ser, Thr, Tyr, Phe, Trp |
| 6 | Gly |
| 7 | His, Lys, Arg, Asp, Glu, Ser, Thr, Tyr, Phe, Trp |
| 8 | Pro, Gly |
| 9 | Cys |
| 10 | Ser, Thr, Asp, Gly |
| 11 | Cit, Gly, Ser, Thr |
| 12 | Cit |
| 13 | Gly |
| 14 | Cys |
| 15 | Arg, Lys, His, Asp, Glu, Ser, Thr, Gln, Asn, Tyr, Phe, Trp |
| 16 | Pro, Gly |
| 17 | Gly |
| 18 | Tyr, His, Lys, Arg, Asp, Glu, Ser, Thr, Gln, Asn, Phe, Trp |

The present invention also relates to shortened versions of type II peptides in which the amino acid residues at position 1 and 2 or at position 1, 2, 3 and 4 are omitted.

According to a further embodiment the present invention relates to a method for producing a peptide described above by classical chemical synthesis, wherein citrulline residues are substituted for arginine residues at certain steps during the chemical synthesis.

According to another embodiment the present invention also relates to a method for producing a peptide described above, wherein the primary amino acid sequence is produced by classical chemical synthesis, and wherein at least one arginine residue subsequently is transformed towards a citrulline residue by contacting said peptide with a peptidylarginine deiminase (PAD).

The peptides of the invention can be prepared by classical chemical synthesis. The synthesis can be carried out in homogeneous solution or in solid phase. For instance, the synthesis technique in homogeneous solution, which can be used, is the one described by Houbenweyl (1974). The peptides of the invention can also be prepared in solid phase according to the methods described by Atherton and Shepard (1989). The forms of the claimed peptides can be obtained by substituting the original arginine residues with citrulline residues during the classical chemical synthesis, or by contacting the peptides after synthesis with a peptidylarginine deiminase of any eukaryotic origin.

The peptides according to this invention can also be prepared by means of recombinant DNA techniques, such as described by Sambrook et al. (1989) or by Stemmer et al. (1995), in prokaryotes or lower or higher eukaryotes. The term ‘lower eukaryote’ refers to host cells such as yeast, fungi and the like. Lower eukaryotes are generally (but not necessarily) unicellular. The term ‘prokaryotes’ refers to hosts such as E.coli, Lactobacillus, Lactococcus, Salmonella, Streptococcus, Bacillus subtilis or Streptomyces. Also these hosts are contemplated within the present invention. Preferred lower eukaryotes are yeast's, particularly species within Schizosaccharomyces, Saccharomyces, Kluiveromyces, Pichia (e.g. Pichia pastoris), Hansenula (e.g. Hansenula polymorpha), Schwaniomyces, Schizosaccharomyces, Yarowia, Zygosaccharomyces and the like. Saccharomyces cerevisiae, S. carlsbergensis and K. lactis are the most commonly used yeast hosts, and are convenient fungal hosts. The term ‘higher eukaryote’ refers to host cells derived from higher animals, such as mammals, reptiles, insects, and the like. Presently preferred higher eukaryote host cells are derived from Chinese hamster (e.g. CHO), monkey (e.g. COS and Vero cells), baby hamster kidney (BHK), pig kidney (PK15), rabbit kidney 13 cells (RK13), the human osteosarcoma cell line 143 B, the human cell line HeLa and human hepatoma cell lines like Hep G2, and insect cell lines (e.g. Spodoptera frugiperda). The host cells may be provided in suspension or flask cultures, tissue cultures, organ cultures and the like. Alternatively the host cells may also be transgenic animals.

According to a further embodiment the present invention also relates to an antibody raised upon immunisation with any of the above-mentioned peptides, with said antibody being specifically reactive with said peptides. The present invention also relates to an anti-idiotype antibody raised upon immunisation with any antibody as defined above, with said anti-idiotype antibody being specifically reactive with said antibody, thereby mimicking any of the above mentioned peptides. These antibodies may be polyclonal or monoclonal.

To prepare antibodies a host animal is immunised using the peptides of the present invention in a pharmaceutically acceptable carrier. Pharmaceutically acceptable carriers include any carrier that does not itself induce the production of antibodies harmful to the individual receiving the composition. Suitable carriers are typically large, slowly metabolised macromolecules such as proteins, polysaccharides, polylactic acids, polyglycolic acids, polymeric amino acids, amino acid copolymers; and inactive virus particles. Such carriers are well known to those of ordinary skill in the art.

Preferred adjuvants to enhance effectiveness of the composition include, but are not limited to: aluminim hydroxide (alum), N-acetyl-muramyl-L-threonyl-D-isoglutamine (thr-MDP) as found in U.S. Pat. No. 4,606,918, N-acetyl-normuramyl-L-alanyl-D-isoglutamine (nor-MDP), N-acetylmuramyl-L-alanyl-D-isoglutaminyl-L-alanine-2-(1′-2′-dipalmitoyl-sn-glycero-3-hydroxyphosphoryloxy)-ethylamine (MTP-PE) and RIBI, which contains three components extracted from bacteria, monophosphoryl lipid A, trehalose dimycolate, and cell wall skeleton (MPL+TDM+CWS) in a 2% squalene/Tween 80 emulsion. Any of the 3 components MPL, TDM or CWS may also be used alone or combined 2 by 2. Additionally, adjuvants such as Stimulon (Cambridge Bioscience, Worcester, MA) or SAF-1 (Syntex) may be used. Further, Complete Freund's Adjuvant (CFA) and Incomplete Freund's Adjuvant (IFA) may be used for non-human applications and research purposes.

The immunogenic compositions typically will contain pharmaceutically acceptable vehicles, such as water, saline, glycerol, ethanol, etc. Additionally, auxiliary substances, such as wetting or emulsifying agents, pH buffering substances, preservatives, and the like, may be included in such vehicles.

Typically, the immunogenic compositions are prepared as injectables, either as liquid solutions or suspensions; solid forms suitable for solution in, or suspension in, liquid vehicles prior to injection may also be prepared. The preparation also may be emulsified or encapsulated in liposomes for enhanced adjuvant effect. The proteins may also be incorporated into Immune Stimulating Complexes together with saponins, for example Quil A (ISCOMS).

Immunogenic compositions used to raise antibodies comprise a ‘sufficient amount’ or ‘an immunologically effective amount’ of the peptides of the present invention, as well as any other of the above mentioned components, as needed. ‘Immunologically effective amount’, means that the administration of that amount to an individual, either in a single dose or as part of a series, is effective to provoke an immune response and to raise antibodies, as defined above. This amount varies depending upon the health and physical condition of the individual, the taxonomic group of the individual to be treated (e.g. nonhuman primate, primate, rabbit, etc.), the capacity of the individual's immune system to synthesise antibodies, the immunogenicity of the antigenic peptide, and its mode of administration, and other relevant factors. It is expected that the amount will fall in a relatively broad range that can be determined through routine trials. Usually, the amount will vary from 0.01 to 1000 mg/dose, more particularly from 0.1 to 100 mg/dose.

The immunogenic compositions are conventionally administered parenterally, typically by injection, for example, subcutaneously or intramuscularly. Additional formulations suitable for other methods of administration include oral formulations and suppositories. Dosage treatment may be a single dose schedule or a multiple dose schedule. The vaccine may be administered in conjunction with other immunoregulatory agents.

The host serum or plasma is collected following an appropriate time interval to provide a composition comprising antibodies reactive with the peptides of the present invention. The gamma globulin fraction or the IgG antibodies can be obtained, for example, by use of saturated ammonium sulfate or DEAE Sephadex, or other techniques known to those skilled in the art. The antibodies are substantially free of many of the adverse side effects, which may be associated with other anti-viral agents such as drugs, for the treatment of infectious, chronic, or recurrent mononucleosis. Such antibodies may also be used to diagnose certain diseases, such as Burkitt's lymphoma, wherein Epstein-Barr virus has been implicated.

The term ‘immunogenic’ refers to the ability of a substance to cause a humoral and/or cellular response, whether alone or when linked to a carrier, in the presence or absence of an adjuvant.

The antibodies of the claimed invention may also be monoclonals that are prepared with said antibody being specifically reactive with any of said peptides, and with said antibody being preferably a monoclonal antibody.

The monoclonal antibodies of the invention can be produced by any hybridoma liable to be formed according to classical methods from spleen cells of an animal, particularly from a mouse or rat, immunised against the claimed peptides of the present invention on the one hand, and of cells of a myeloma cell line on the other hand, and to be selected by the ability of the hybridoma to produce the monoclonal antibodies recognising the citrullinated forms of the peptides which has been initially used for the immunisation of the animals.

The antibodies involved in the invention can be labelled by an appropriate label of the enzymatic, fluorescent, or radioactive type.

The monoclonal antibodies according to this preferred embodiment of the invention may be humanised versions of mouse monoclonal antibodies made by means of recombinant DNA technology, departing from parts of mouse and/or human genomic DNA sequences coding for H and L chains or from cDNA clones coding for H and L chains.

Alternatively the monoclonal antibodies according to this preferred embodiment of the invention may be human monoclonal antibodies. These antibodies according to the present embodiment of the invention can also be derived from human peripheral blood lymphocytes of patients with rheumatoid arthritis. Such human monoclonal antibodies are prepared, for instance, by means of human peripheral blood lymphocytes (PBL) repopulation of severe combined immune deficiency (SCID) mice (for recent review, see Duchosal et al. 1992) or by screening vaccinated individuals for the presence of reactive B-cells by means of the antigens of the present invention.

The present invention also relates to truncated versions or single chain versions of the antibodies and anti-idiotype antibodies as defined above that have retained their original specificity for reacting with the antigens.

The present invention also relates to a method for detecting antibodies that specifically react with the peptides or anti-idiotype antibodies of the present invention, present in a biological sample, comprising:

(i) contacting the biological sample to be analysed for the presence of said antibodies with a peptide or anti-idiotype antibody as defined above,

(ii) detecting the immunological complex formed between said antibodies and said peptide or anti-idiotype antibody.

The present invention also relates to a reverse method for detecting the peptides and/or the anti-idiotype antibodies of the present invention with antibodies present in a biological sample that specifically react with said peptides and/or anti-idiotype antibodies that mimic such peptides, comprising:

(i) contacting the biological sample to be analysed for the presence of said peptides or anti-idiotype antibodies with the antibodies as defined above,

(ii) detecting the immunological complex formed between said antibodies and said peptide or anti-idiotype antibody.

The methods as defined above, can be used in the diagnosis of rheumatoid arthritis.

The present invention also relates to a diagnostic kit for use in detecting autoimmune diseases such as rheumatoid arthritis wherein said kit comprises at least one of the above mentioned peptides or antibodies, and with said peptide or antibody being possibly bound to a solid support.

More preferably said kit is comprising a range of said peptides or said antibodies, possibly in combination with other epitopes that can characterise autoimmune diseases, wherein said peptides and/or epitopes are attached to specific locations on a solid substrate. More preferably said solid support is a membrane strip and said peptides are coupled to the membrane in the form of parallel lines. It has to be understood that certain peptides or antibodies as defined above, alternatively, are not attached to a solid support but are provided in the binding solution to be used as competitors and/or to block other antibodies that are present in sera from patients with autoimmune diseases other than rheumatoid arthritis, thereby decreasing or eliminating possible cross-reaction and/or a-specific binding.

According to a specific embodiment, the present invention relates to the development of a diagnostic technique that allows differentiation between those autoimmune diseases in which the characteristic antibodies often crossreact with the same antigen, thus resulting in difficult and slow diagnosis. Such diagnostic technique can be obtained by the simultaneous use of several antigens and/or anti-idiotype antibodies of the present invention.

The present invention also relates to a diagnostic kit for use in detecting the presence of said antibodies, said kit comprising at least one peptide or anti-idiotype antibody as defined above, with said peptide or anti-idiotype antibody being preferably bound to a solid support.

The present invention also relates to a diagnostic kit for determining the type of autoimmune disease, said kit comprising at least one peptide or anti-idiotype antibody as defined above, with said peptide or anti-idiotype antibody being preferably bound to a solid support.

The present invention also relates to a diagnostic kit as defined above, said kit comprising a range of said peptides and/or anti-idiotype antibodies, which are attached to specific locations on a solid substrate.

The present invention also relates to a diagnostic kit as defined above, wherein said solid support is a membrane strip and said peptides and/or anti-idiotype antibodies are coupled to the membrane in the form of parallel lines.

The immunoassay methods according to the present invention may utilise for instance single or specific oligomeric antigens, dimeric antigens, as well as combinations of single or specific oligomeric antigens. The peptides of the present invention may be employed in virtually any assay format that employs a known antigen to detect antibodies that characterise a certain disease or infection. A common feature of all of these assays is that the antigenic peptide or anti-idiotype antibody is contacted with the body component suspected of containing the antibodies under conditions that permit the antigen to bind to any such antibody present in the component. Such conditions will typically be physiologic temperature, pH and ionic strength using an excess of antigen. The incubation of the antigen with the specimen is followed by detection of immune complexes comprised of the antigen.

Design of the immunoassays is subject to a great deal of variation, and many formats are known in the art. Protocols may, for example, use solid supports, or immunoprecipitation. Most assays involve the use of labelled antibody or peptide; the labels may be, for example, enzymatic, fluorescent, chemiluminescent, radioactive, or dye molecules. Assays, which amplify the signals from the immune complex, are also known; examples of which are assays, which utilise biotin and avidin or streptavidin, and enzyme-labelled and mediated immunoassays, such as ELISA assays.

The immunoassay may be, without limitation, in a heterogeneous or in a homogeneous format, and of a standard or competitive type. In a heterogeneous format, the peptide or anti-idiotype antibody is typically bound to a solid matrix or support to facilitate separation of the sample from the peptide or anti-idiotype antibody or microprotein after incubation. Examples of solid supports that can be used are nitrocellulose (e.g., in membrane or microtiter well form), polyvinyl chloride (e.g., in sheets or microtiter wells), polystyrene latex (e.g., in beads or microtiter plates, polyvinylidene fluoride (known as Immunolon™), diazotized paper, nylon membranes, activated beads, and Protein A beads. For example, Dynatech Immunolon™ 1 or Immunolon™ 2 microtiter plates or 0.25-inch polystyrene beads (Precision Plastic Ball) can be used in the heterogeneous format. The solid support containing the antigenic peptides or anti-idiotype antibodies is typically washed after separating it from the test sample, and prior to detection of bound antibodies. Both standard and competitive formats are known in the art.

In a homogeneous format, the test sample is incubated with the combination of antigens in solution. For example, it may be under conditions that will precipitate any antigen-antibody or anti-idiotype antibody-antibody complexes, which are formed. Both standard and competitive formats for these assays are known in the art. For instance, to characterise rheumatoid arthritis in a standard format, the amount of rheumatoid arthritis antibodies in the antibody-antigen complexes is directly monitored. This may be accomplished by determining whether a second type of labelled anti-xenogenetic (e.g. anti-human) antibodies, which recognise an epitope on the first type of rheumatoid arthritis-antibodies, will bind due to complex formation. In a competitive format, the amount of rheumatoid arthritis-antibodies in the sample is deduced by monitoring the competitive effect on the binding of a known amount of labelled antibody (or other competing ligand) in the complex. The detection of rheumatoid arthritis-antibodies for diagnosis of rheumatoid arthritis is used as an illustration. Wherever the term ‘rheumatoid arthritis-antibodies’ is used throughout the specification, this should not be considered as limitative. Like wise, the other autoimmune diseases are diagnosed by detection of other antibodies, and mononucleosis is diagnosed by detection of anti-Epstein-Barr virus antibodies.

Complexes formed comprising rheumatoid arthritis-antibody (or in the case of competitive assays, the amount of competing antibody) are detected by any of a number of known techniques, depending on the format. For example, unlabelled rheumatoid arthritis-antibodies in the complex may be detected using a conjugate of anti-xenogenetic Ig complexed with a label (e.g. an enzyme label).

In an immunoprecipitation or agglutination assay format the reaction between the rheumatoid arthritis-antigens and the rheumatoid arthritis-antibody forms a network that precipitates from the solution or suspension and forms a visible layer or film of precipitate. If no rheumatoid arthritis-antibody is present in the test specimen, no visible precipitate is formed.

Currently, there exist three specific types of particle agglutination (PA) assays. These assays are used for the detection of antibodies to various antigens when coated to a support. One type of this assay is the hemagglutination assay using red blood cells (RBCs) that are sensitised by passively adsorbing antigen (or antibody) to the RBC. The addition of specific antigen antibodies present in the body component, if any, causes the RBCs coated with the purified antigen to agglutinate.

To eliminate potential non-specific reactions in the hemagglutination assay, two artificial carriers may be used instead of RBC in the PA. The most common of these are latex particles. However, gelatin particles may also be used. The assays utilising either of these carriers are based on passive agglutination of the particles coated with purified antigens.

The antigenic peptides of the present invention will typically be packaged in the form of a kit for use in these immunoassays. The kit will normally contain in separate containers the antigenic peptide or anti-idiotype antibody, control antibody formulations (positive and/or negative), labelled antibody when the assay format requires the same and signal generating reagents (e.g. enzyme substrate) if the label does not generate a signal directly. The antigenic peptide or anti-idiotype antibody may be already bound to a solid matrix or separate with reagents for binding it to the matrix. Instructions (e.g. written, tape, CD-ROM, etc.) for carrying out the assay usually will be included in the kit.

The solid phase selected can include polymeric or glass beads, nitrocellulose, microparticles, and microwells of a reaction tray, test tubes and magnetic beads. The signal-generating compound can include an enzyme, a luminescent compound, a chromogen, a radioactive element and a chemiluminescent compound. Examples of enzymes include alkaline phosphatase, horseradish peroxidase and beta-galactosidase. Examples of enhancer compounds include biotin, anti-biotin and avidin. In order to block the effects of rheumatoid factor-like substances, the test sample is subjected to conditions sufficient to block the effect of rheumatoid factor-like substances. These conditions is comprise contacting the test sample with a quantity of for instance Rabbit Ig or anti-human IgG, preferably aggregated, to form a mixture, and incubating the mixture for a time and under conditions sufficient to form a reaction mixture product substantially free of rheumatoid factor-like substance.

The present invention particularly relates to an immunoassay format in which several peptides of the invention are coupled to a membrane in the form of parallel lines. This assay format is particularly advantageous for allowing discrimination between the separate autoimmune diseases.

According to another embodiment the present invention relates to an immunotoxin molecule comprising a cell recognition molecule being a peptide or an antibody as described above covalently bound to a toxin molecule or active fragment thereof.

The present invention also relates to a peptide or an antibody or an immunotoxin molecule as described above or a composition thereof for use as a medicament.

The present invention further relates to a peptide or an antibody or an immunotoxin molecule as described above or a composition thereof for the preparation of a medicament or of a diagnosticum for rheumatoid arthritis.

The present invention also relates to the use of a peptide described above or a composition thereof for the preparation of a medicament to treat autoimmune diseases by increasing the size of antigen-immune complexes, thereby improving the clearance of the formed immune complexes.

The terms “a pharmaceutical composition for treating” or “a drug or medicament for treating” or “use of proteins for the manufacture of a medicament for the treatment” relate to a composition comprising any peptide as described above or any antibody specifically binding to these peptides and a pharmaceutically acceptable carrier or excipient (both terms can be used interchangeably) to treat diseases as described above. Suitable carriers or excipients known to the skilled man are saline, Ringer's solution, dextrose solution, Hank's solution, fixed oils, ethyl oleate, 5% dextrose in saline, substances that enhance isotonicity and chemical stability, buffers and preservatives. Other suitable carriers include any carrier that does not itself induce the production of antibodies harmful to the individual receiving the composition such as proteins, polysaccharides, polyactic acids, polyglycolic acids, polymeric amino acids and amino acid copolymers. The “medicament” may be administered by any suitable method within the knowledge of the skilled man.

The present invention also relates to the use of a peptide described above or a composition thereof for the preparation of a medicament for oral or nasal administration to treat autoimmune diseases by inducing a state of systemic hyporesponsiveness or tolerance to said peptide or composition.

Another route of administration is parenterally. In parenterally administration, the medicament of this invention will be formulated in a unit dosage injectable form such as a solution, suspension or emulsion, in association with the pharmaceutically acceptable excipients as defined above. However, the dosage and mode of administration will depend on the individual. Generally, the medicament is administered so that the peptide of the present invention is given at a dose between 1 μg/kg and 10 mg/kg, more preferably between 10 μg/kg and 5 mg/kg, most preferably between 0.1 and 2 mg/kg. Preferably, it is given as a bolus dose. Continuous infusion may also be used. If so, the medicament may be infused at a dose between 5 and 20 μg/kg/minute, more preferably between 7 and 15 μg/kg/minute.

### EXAMPLES

### Example 1

### Reactivity of Synthetic Peptides in a Line Immunoassay (LIA) System

1.1 Synthetic Peptides

Biotinylated peptides were synthesised according to standard Fmoc-chemistry described by Houbenweyl (1974) and purified by reversed-phase HPLC using a 8-70% linear gradient of 70% acetonitrile in 0.1% trifluoroacetic acid (TFA). Oxidation was performed in 50-mM sodium carbonate, pH 9.6. For testing of the linear structures, peptides were treated with 5 mM DTT, followed by addition of 35-mM iodoacetamide. Molecular structure was confirmed by MALDI-MS. A list of the different peptides is provided in table 4.

TABLE 4
Amino acid sequences of the different synthetic peptides used for detection of
anti-filaggrin on LIA

| | | | | | | | | | | | | | | | | | | |
|---|---|---|---|---|---|---|---|---|---|---|---|---|---|---|---|---|---|---|
| IGP1611 | Q | D | T | I | H | G | H | P | C | S | X | X | G | H | R | C | G | Y |
| IGP1646 | Q | D | T | I | H | G | H | P | C | S | S | X | G | H | R | C | G | Y |
| IGP1647 | Q | D | T | I | H | G | H | P | C | S | X | X | G | H | Q | C | G | Y |
| IGP1648 | Q | D | T | I | H | G | H | P | C | S | X | X | G | H | R | C | G | Q |
| IGP1649 | Q | D | T | I | H | G | H | P | C | S | X | X | G | H | Q | C | G | Q |
| IGP1650 | Q | D | T | I | H | G | H | P | C | S | X | X | G | C | R | P | G | Y |
| IGP1651 | | | | | H | G | H | P | C | S | X | X | G | H | R | C | G | Y |
| IGP1676 | | | | | H | G | H | P | C | S | X | X | G | C | R | P | G | Y |
| IGP1687 | | | | | H | G | H | G | C | D | X | X | G | H | R | C | G | Q |
| IGP1684 | | | | | H | G | H | G | C | D | S | X | G | H | R | C | G | Q |
| IGP1685 | Q | D | T | I | V | G | W | G | C | D | S | X | G | C | R | P | G | Q |
| IGP1686 | | | | | V | G | W | G | C | D | S | X | G | C | R | P | G | Q |

X: citrulline; C: cysteines that form a disulphide bridge

1.2 LIA Evaluation

Patient sera were obtained from the Department of Rheumatology at the University Hospital in Ghent and fulfilled the ACR criteria for RA. Control sera consisted of sponylarthropathy (SPA), systemic lupus erythematosus (SLE), osteoarthritis and healthy subjects.

Streptavidin-complexed peptides were applied directly on a nylon membrane with a plastic backing. Blocked strips were incubated during 1 hour with human sera diluted 1/100 in 1 ml PBS, 0.5% caseine, 0.1% Triton X705, 10 mM MgCl₂.6H₂O. After washing with PBS, 0.05% Tween20, goat anti-human IgG-AP conjugate (Promega) diluted in PBS, 0.1% caseine, 0.2% Triton X705 was added for 30 min. Strips were developed in 100 mM NaCl, 100 mM Tris-HCl, pH 9.8, 50 mM MgCl₂.6H₂O substrate buffer containing the chromogenic substrate NBT/BCIP. Reaction was stopped after 30 min by addition of 0.2 N H₂SO₄. Air-dried strips were scanned using a HP Scanjet 5P scanner and an in-house LIA interpretation software program.

1.3 Results

Following peptides were evaluated on LIA using 100 RA and 100 disease control sera (11 SPA, 39 SLE, and 500A): IGP1611, IGP1646, IGP1647, IGP1648, IGP1649, IGP1650, and IGP1651. Results are summarised in Table 5.

Statistical processing of the data by discriminant and principal component analysis revealed that peptides IGP1611, IGP1646, IGP1650 and IGP1651 attributed to a maximal variation and highest combined sensitivity. ROC-analysis was performed for each separate line in order to determine sensitivities at each particular specificity level. Individual peptides were able to detect >40% of RA sera at a specificity of 95%.

TABLE 5
LIA-reactivity of 100 RA and 100 disease control sera with citrullinated
synthetic peptides obtained by molecular modelling and computational
chemistry. ROC-analysis was used to determine specificity levels.

| Antigen | 90% specificity level | 95% specificity level |
|---|---|---|
| IGP1611 | 53% | 45% |
| IGP1646 | 53% | 37% |
| IGP1647 | 45% | 34% |
| IGP1648 | 59% | 46% |
| IGP1649 | 45% | 33% |
| IGP1650 | 59% | 44% |
| IGP1651 | 53% | 41% |

To our surprise, IGP1676 was only reactive with two sera when tested on LIA with a battery of 159 RA sera. This peptide was however largely similar to IGP1650, but lacked the first four residues. Upon modelling studies and computational chemistry, it became clear that the three-dimensional structure of this peptide was completely disrupted and presents a deviating three-dimensional structure resulting in an inaccessible citrulline epitope.

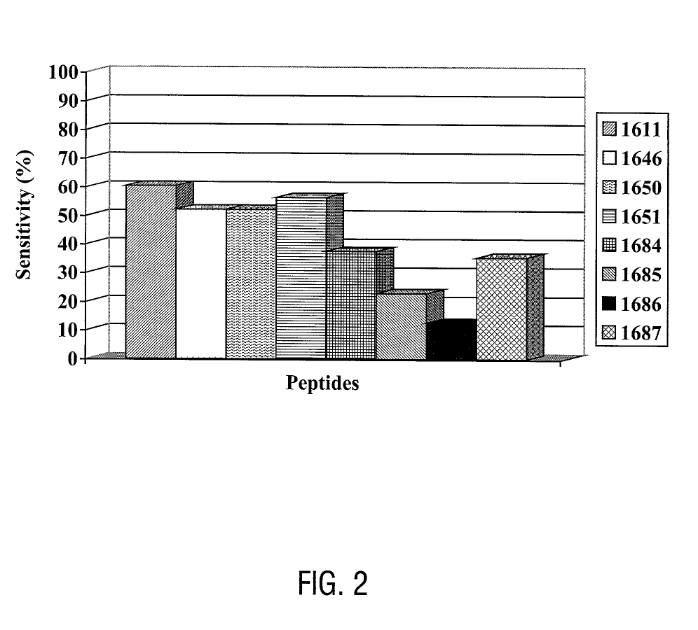

In subsequent experiments, the above-mentioned peptides were evaluated in combination with IGP1684, IGP1685, IGP1686 and 1687, all in their purified form (FIG. 2). Higher sensitivities were obtained for IGP1611-1651 and reached as high as 60% of the RA group tested. The other peptides showed lower reactivity and were not complementary to the first set of peptides.

### Example 2

### Importance of Cyclisation of Synthetic Peptides for Immunoreactivity

2.1 Cyclisation and Linearisation of Peptides

HPLC-fractions containing correct synthetic peptides were vacuum-dried, resuspended in 50-mM carbonate buffer, pH 9.6 at 200 μg/ml and oxidised by vigorous stirring. MALDI measurements confirmed that the peptides were predominantly present in their oxidised form.

For linearisation, dried peptides were solubilized in MQ-water at 1 mg/ml and treated with 5 mM DTT during 3h at 37° C. Part of the material was subsequently incubated with 35 mM iodoacetamide for 30 min at 37° C. in order to block all sulfhydryl groups and so to prevent partial reoxidation of the peptides on the membrane.

2.2 Results

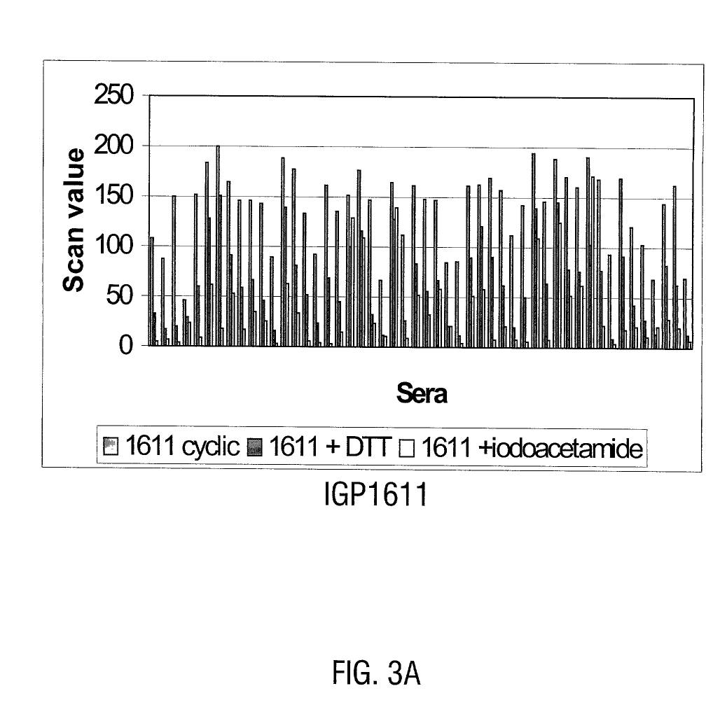

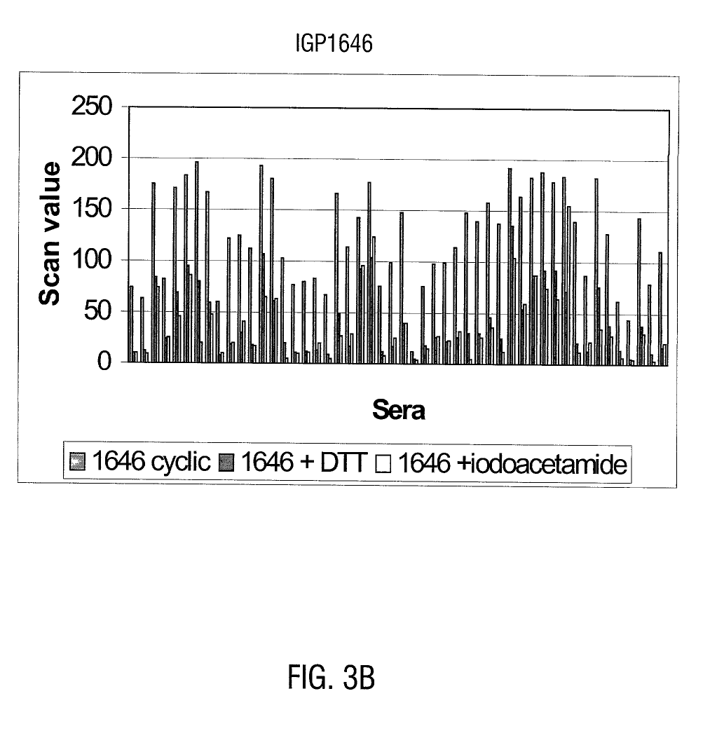

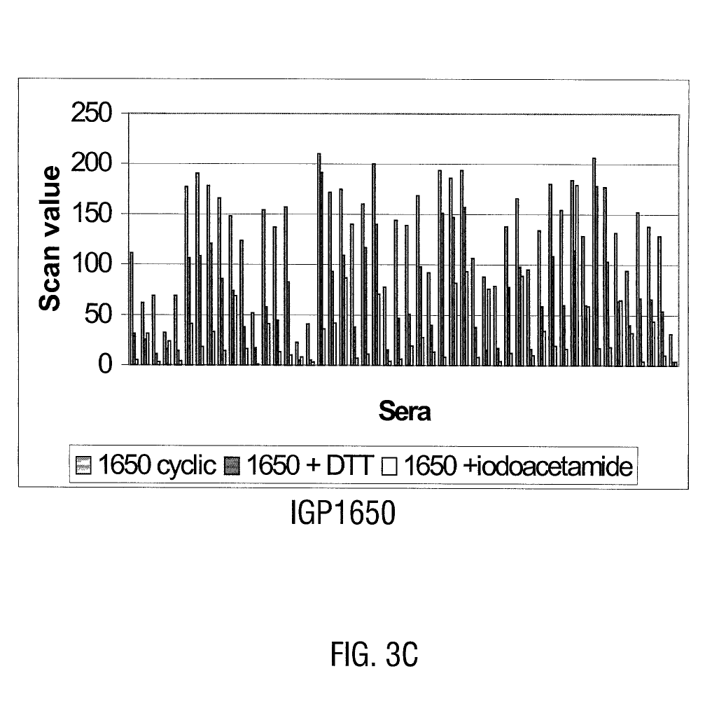

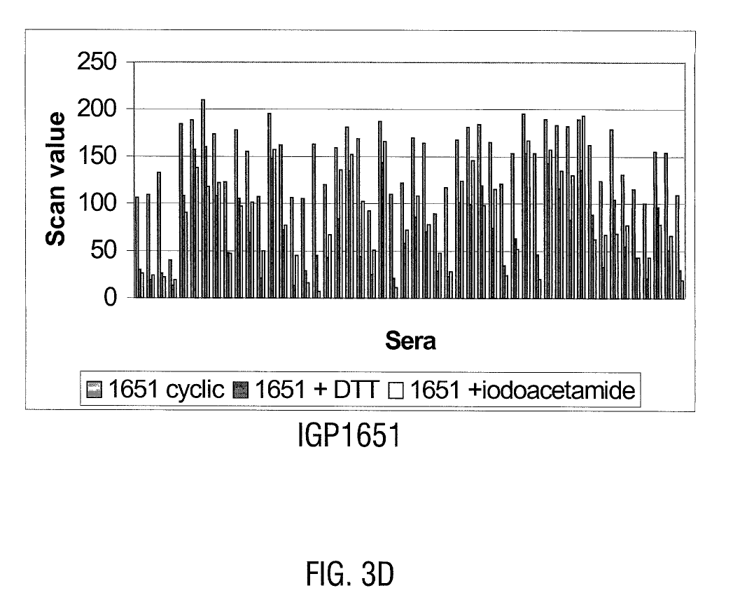

Peptides IGP1611, 1646, 1650 and 1651 were applied on strips both in their cyclic or linear forms and evaluated with 50 RA sera. Results for the individual peptides are depicted in FIGS. 3(a-d). It was clear that linearisation by DTT treatment or blocking of cysteines with iodoacetamide resulted in much lower reactivities. In the latter condition, only an average of 24, 26, 23 and 52% of the original scan value was recovered for the respective peptides (data not shown). As there are considerable inter-patient variation, rather large SD's were obtained. However, from these results it is obvious that linear structures as being presented here are not useful for detection of anti-filaggrin antibodies in human sera in a LIA test system.

### EXAMPLE 3

### Comparison of Synthetic Peptides with Existing Peptides and Reference Test Systems

3.1 Western Blotting Using Natural Filaggrin

Natural filaggrin was purified from human skin obtained freshly after abdominoplasty according to the protocol of Simon et al. (1993). The amount of protein was determined by the Bradford protein assay as modified by Peterson (1983) using BSA standard curves. The crude filaggrin preparation was subjected to 15% Laemmli SDS-PAGE using the Bio-Rad mini-gel apparatus. Two-μg protein/cm was loaded in a large slot and electrophoresed under standard conditions. The gel was subsequently electroblotted onto nitrocellulose membrane in 10% methanol, 10 mM CAPS, pH 11.0 during 40 min. The blot was blocked in PBS, 0.05% Tween20, 1% gelatin and cut into 3 mm strips, which were probed with human sera overnight at a 1/100 dilution in PBS, 0.05% Tween20, 0.1% gelatin. As a secondary antibody the anti-human IgG-AP conjugate (Sigma, St Louis, MI) was added at a 1/1000 dilution; visualisation occurred with the NBT/BCIP chromogenic substrate.

3.2 Preparation of Citrullinated Recombinant Filaggrin

Recombinant human filaggrin was expressed in E. coli and purified by Ni-IMAC chromatography. Citrullination was performed as described by Senshu et al. (1995) using peptidylarginine deiminase (Sigma) at a 1/120 enzyme/substrate ratio during 4h at 37° C.

3.3 APF Test

Anti-perinuclear factor (APF) was determined by incubation of mouth mucosa cells from positive donors with patient sera as described by Janssens et al. (1988).

3.4 Results

Three synthetic peptides (IGP1475, 1477 and 1564) described by Girbal-Neuhauser (1999) (patent application WO 99/35167) and the cyclic peptide IGP1546 described in the patent application WO 98/22503, were synthesised and used for comparison with IGP1611, 1646, 1650 and 1651 on LIA. Structures are shown in Table 6.

TABLE 6
Amino acid sequences of the synthetic peptides described in the patent
applications WO 99/35167 and WO 98/22503 used for detection of RA specific
autoantibodies in human sera.

| | | | | | | | | | | | | | | | | | | | | | | |
|---|---|---|---|---|---|---|---|---|---|---|---|---|---|---|---|---|---|---|---|---|---|---|
| IGP1475 | T | G | S | S | T | G | G | X | Q | G | S | H | H | E | | | | | | | | |
| IGP1477 | E | S | S | R | D | G | S | X | H | P | R | S | H | D | | | | | | | | |
| IGP1564 | E | Q | S | A | D | S | S | X | H | S | G | S | G | H | | | | | | | | |
| IGP1546 | S | H | Q | C | H | Q | E | S | T | X | G | R | S | R | G | R | C | G | R | S | G | S |

X: citrulline

On the same strips, also citrullinated recombinant E. coli filaggrin was applied. In total, 212 established RA sera fulfilling the ACR criteria and 153 disease control sera selected from the daily rheumatology clinic because of a clinical presentation comparable to RA were used for evaluation. Diagnosis different from RA in the control group was assigned 6 months after inclusion. LIA strips were scanned so that scan values could be used for ROC analysis and determination of specificity/sensitivity levels. Table 7 shows the sensitivities obtained for each line in the RA serum group at cut-off values resulting in 98% specificity.

ROC-analysis was used to determine the 98% specificity level. Any of the combination of lines resulted in a specificity of 96%.

None of the described peptides IGP1475, 1477, 1564 or 1546 yielded better results. Highest sensitivities were obtained with IGP1650 and 1651. Due to partial complementary reactivity, 66% of the sera could be detected when taking into account different antigen lines IGP1650, 1651 and recombinant filaggrin, while specificity remained as high as 96%.

TABLE 7
LIA-reactivity of 225 RA and 154 disease control sera with citrullinated
synthetic peptides described in the patent applications WO 99/35167 and
WO 98/22503.

| | Sensitivity | Combined |
|---|---|---|
| Antigen line | at 98% spec. level | sensitivity |
| Citr. rec. filaggrin | 32% | |
| IGP1611 | 55.7% | |
| IGP1646 | 51.0% | |
| IGP1650 | 58.5% | |
| IGP1651 | 58.5% | |
| IGP1475 | 7.5% | |
| IGP1477 | 9.0% | |
| IGP1564 | 13.0% | |
| IGP1546 | 53.0% | |
| Rec. fg or 1650 | | 63.7% |
| Rec. fg or 1650 or 1651 | | 66.0% |
| Rec. fg or 1611 or 1646 or 1650 or 1651 | | 67.0% |

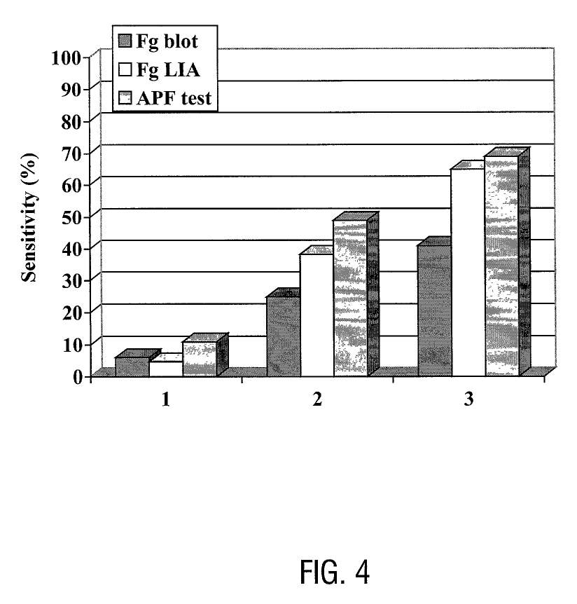

LIA-results were further compared to Western blot with natural filaggrin and to the APF fluorescence test using human buccal cells (FIG. 4). Both 212 longstanding RA sera, 153 disease controls as well as 47 sera from patients with early disease (less than 12 months of symptoms) were included. Western blot performed less sensitive in the two RA groups: 41% and 25% resp. versus 66% and 38% resp. for LIA. Furthermore, somewhat lower specificity of 94% was obtained for the Western blot system. In contrast, the APF test yielded higher sensitivities of 69% and 49% resp., but at a significant lower specificity of 89% however. Nevertheless, as the APF test is hardly used in routine labs due to its user-unfriendliness, the LIA using the peptides of the present invention, offers an excellent alternative for determination of anti-filaggrin antibodies in human sera. Comparison between LIA and anti-filaggrin determination on Western blot showed a moderate agreement with a Cohen's Kappa=0.554 (95% CI 0.455-0.6532), while between LIA and APF a substantial agreement was observed with a Cohen's Kappa=0.693 (95% CI 0.661-0.784).

### Example 4

Performance testing of a line immunoassay (LIA) containing individual markers (in vitro citrullinated recombinant filaggrin and two citrullinated synthetic peptides (IGP1650 and IGP1651)) in RA patients and patients with other rheumatologic disorders.

4.1 Line Immunoassay

A line immunoassay was developed as described in example 1 containing as individual antigen lines two citrullinated synthetic peptides IGP1650 and IGP1651 and citrullinated recombinant filaggrin. This LIA was evaluated using 335 sera from individual RA patients and 254 control sera derived from patients with non-RA rheumatological disorders (see table 8).

TABLE 8

| Diagnosis | Number of samples |
|---|---|
| Myositis | 15 |
| OA | 30 |
| PMR | 1 |
| Sjögren | 15 |
| SLE/MCTD | 90 |
| SpA | 50 |
| SSc | 53 |
| Total | 254 |

Control sera (OA = Osteo-arthritis; SLE = Systemic Lupus Erythematosis; MCTD = Mixed Connective Tissue Disease; SSc = Scleroderma; PMR = Polymyalgia Rheumatica; SpA = Spondylo-arthritis (containing Psoriatic arthritis, REA and SpA))

4.2 Reference Serum Tests: RF, Anti-Keratin Antibodies (AKA) and Anti-RA33

RF was measured by both nephelometric methods and ELISA (Imtec, Germany). AKA were determined by immunofluorescence tests on rat oesophagus as described (Vincent et al., 1999). Determination of anti-RA33 reactivity was performed by immunoblot (Hassfeld et al., 1995)

4.3 Results

The performance of the LIA RA will be discussed based on two versions of the test:

LIA3=LIA taking into account all 3 antigen lines for the interpretation; that is, the test is considered positive if at least one of the antigen lines is reactive.

LIA2=LIA taking into account only 2 antigen lines (IGP1650 & IGP1651) for the interpretation; that is, the test is considered positive if at least one of these two antigen lines is reactive.

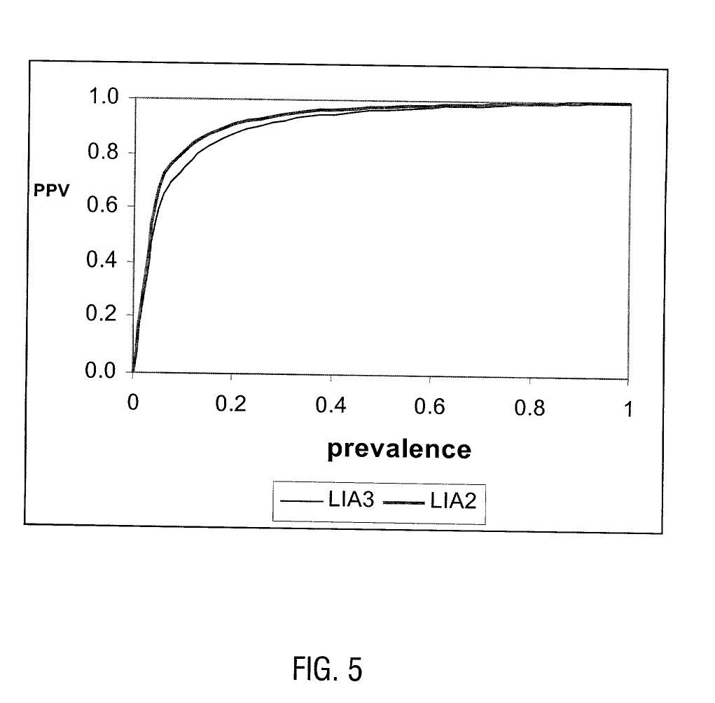

The overall sensitivity of LIA3 is 65.1% versus 61.8% if only the reactivity towards the citrullinated peptides (LIA2) is considered. The overall specificity of LIA3 is 97.6% versus 98.4% for LIA2. Based on a prevalence of rheumatoid arthritis patients of 20% in a population that visits a rheumatologist, the LIA with 3 lines has a PPV (positive predictive value) of 87.3% whereas the LIA with the peptides only, has a PPV of 90.7% (FIG. 5).

Because selection in this study group has been biased due to the fact that i) RF positivity is an ACR criterion for RA, ii) the daily rheumatological practice is not represented and iii) the controls were selected in order to challenge the LIA test, direct comparison between LIA and RF test as for sensitivity and specificity is not appropriate. Nevertheless, a LIA2 positivity of 37% was observed in selected RF negative samples, demonstrating a real complementarity to the RF-test (Table 9). Of additional importance was that all RF negative disease controls proved also anti-filaggrin negative. Furthermore, 52 out of 55 RF false positive controls were negative for LIA2.

TABLE 9
Determination of RF and anti-filaggrin antibodies in human sera.

| | LIA2 | RA group | Disease controls |
|---|---|---|---|
| | Anti-filaggrin | n = 333 | N = 189 |
| − | − | 51 | 134 |
| | + | 30 | − |
| + | − | 76 | 52 |
| | + | 176 | 3 |

Anti-keratin antibodies (AKA), also targeting citrullinated filaggrin epitopes, were analyzed in a group of 231 RA sera (Table 10). A good agreement between both tests was observed (Cohen's Kappa value of 0.732; 95% CI 0.641-0.824), whereby a slightly more sensitive LIA was noticed, without statistical significance however.

TABLE 10
Comparison between AKA and anti-filaggrin LIA2 determination in 231
individual RA sera.

| | LIA2 | RA group |
|---|---|---|
| AKA | Anti-filaggrin | N = 231 |
| − | − | 72 |
| | + | 19 |
| + | − | 10 |
| | + | 130 |

Another marker described in literature as being present in RA sera consists of anti-RA33 antibodies. As can be seen in Table 11, an appreciable higher sensitivity (52% versus 24%) and specificity (98% versus 91%) was observed for LIA2.

TABLE 11
Determination of anti-RA33 and anti-filaggrin antibodies.

| | LIA2 | RA group | Disease controls |
|---|---|---|---|
| Anti-RA33 | Anti-filaggrin | N = 210 | n = 155 |
| − | − | 77 | 138 |
| | + | 82 | 3 |
| + | − | 24 | 14 |
| | + | 27 | − |

In conclusion, anti-filaggrin antibodies can be retrieved by a test based on citrullinated peptides only. This LIA2 test has a high specificity and thus a high PPV, and shows clear complementarity to RF. As it can replace AKA testing, it is a valuable tool in the differential diagnosis of RA.

### REFERENCES

The following references, to the extent that they provide exemplary procedural or other details supplementary to those set forth herein, are specifically incorporated herein by reference.

Atherton and Shepard in “Solid phase peptide synthesis” IRL Press, Oxford, UK, 1989.

Boers, M., Verhoeven, A. C., Markusse, H. M., van de Laar, M. A. F. J., Westhovens, R., van Denderen, J. C., van Zebben, D., Dijkmans, B. A. C., Peeters, A. J. and Jacobs, P. ‘Randomised comparison of combined step-down prednisolone, methotrexate and sulpasalazine with sulphasalazine alone in early rheumatoid arthritis. Lancet 350:309, 1997.

Duchosal, M. A., Eming, S. A., Fischer, P., Leturcq, D., Barbas, C. F. d., McConahey, P. J., Caothien, R. H., Thornton, G. B., Dixon, F. J. and Burton, D. R. ‘Immunisation of hu-PBL-SCID mice and the rescue of human monoclonal Fab fragments through combinatorial libraries.’ Nature 355:258, 1992.

Girbal-Neuhauser, E., Durieux, J. -J., Arnaud, M., Dalbon, P., Sebbag, M., Vincent, C., Simon, M., Senshu, T., Masson-Bessiere, C., Jolivet-Reynaud, C., Jolivet, M. and Serre, G. ‘The epitopes targeted by the rheumatoid arthritis-associated antifilaggrin autoantibodies are posttranslationally generated on various sites of (pro)filaggrin by deimination of arginine residues’ J. of Immunology, 162: 585-594, 1999

Hassfeld, W., Steiner, G., Studnicka-Benke, A., Skriner, K., Graninger, W., Fischer, I., Smolen, J. -S. ‘Autoimmune response to components of the spliceosome—an immunological link between rheumatoid arthritis, mixed connective tissue disease and systemic lupus erythematosus’ Arthritis Rheum, 38: 777-785, 1995.

Houbenweyl in “Methode der Organischen Chemie” edited by E. Wunsch, vol 15-I et II, Thieme, Stuttgart, Germany, 1974.

Janssens, X., Veys, E. M., Verbruggen, G. and Declercq, L. ‘The diagnostic significance of the antiperinuclear factor for rheumatoid arthritis’ J. Rheumatol, 15:1346-1350, 1988.

Palosuo, T., Lukka, M., Alenius, H., Kalkkinen, N., Aho, K., Kurki, P., Heikkila, R., Nykänen, M. and von Essen, R. ‘Purification of filaggrin from human epidermis and measurement of antifilaggrin autoantibodies in sera from patients with rheumatoid arthritis by an Enzyme-Linked Immunosorbent Assay.’ Int Arch Allergy Immunol, 115:294-302, 1998.

Peterson, G. L. ‘Determination of total protein’ Meth. Enzymol, 91:95-119, 1983

Sambrook, J., Fritsch, E. F. and Maniatis, T. ‘Molecular Cloning, a laboratory manual, second edition. Cold Spring Harbor University Press, Cold Spring Harbor, N.Y. USA, 1989.

Schellekens, G. A., de Jong, B. A. W., van den Hoogen, F. H. J., van de Putte, L. B. A. and van Venrooij, W. J. ‘Citrulline is an essential constituent of antigenic determinants recognised by rheumatoid arthritis-specific autoantibodies.’ J. Clin. Invest. 101:273, 1998

Senshu, T., Akiyama, K., Kan, S., Asaga, H., Ishigami, A. and Manabe, M. ‘Detection of deiminated proteins in rat skin: probing with a monospecific antibody after modification of citrulline residues.’ J. Invest. Dermatol. 105:163, 1995

Simon, M., Girbal, E., Sebbag, M., Gomés-Daudrix, V., Vincent, C., Salama, G. and Serre, G. ‘The cytokeratin filament-aggregating protein filaggrin is the target of the so-called ‘antikeratin antibodies’, autoantibodies specific for rheumatoid arthritis.’ J. Clin. Invest., 92:1387-1393, 1993.

Stemmer, W. P., Crameri, A., Ha, K. D., Brennan, T. M. and Heynecker, H. L. ‘Single step assembly of a gene and entire plasmid from large numbers of oligodeoxyribonucleotides’ Gene 164:49-53, 1995.

Vincent, C., de Keyser, F., Masson-Bessiére, C., Sebbag, M., Veys, E. M. and Serre, G. ‘Anti-perinuclear factor compared with the so called “antikeratin” antibodies and antibodies to human epidermis filaggrin, in the diagnosis of arthritides.’ Ann. Rheum. Dis. 58:42-48, 1999.

What is claimed is:

1. A peptide comprising a sequence of less than 50 amino acids characterised in that
it contains a peptide turn comprising at least one citrulline residue, and
it contains less than 12 amino acids between two cysteine residues, with said citrulline residue being one of the amino acids between said cysteine residues and
said peptide is specifically recognised by autoimmune antibodies from patients suffering from rheumatoid arthritis.

2. A peptide according to claim 1 characterised in that said peptide is a cyclic peptide.

3. A peptide according to claim 1-2 characterised in that said peptide is biotinylated.

4. A peptide according to claim 1-3 characterised in that said peptide is a synthetic peptide.

5. A peptide according to claim 1-4 characterised in that said peptide contains 4 or 6 residues between the cysteine residues.

6. A peptide according to claim 1-5 characterised in that said peptide has a sequence containing 14, 15, 16, 17 or 18 amino acids.

7. A peptide according to claim 1-6 characterised in that said peptide has one of the following primary amino acid structures:
8 AA—Cysteine—2 AA—Citrulline—3 AA—Cysteine—2 AA, or
5 AA—Cysteine—2 AA—Citrulline—3 AA—Cysteine—2 AA, or
4 AA—Cysteine—2 AA—Citrulline—3 AA—Cysteine—2 AA, or
8 AA—Cysteine—2 AA—Citrulline—1 AA—Cysteine—4 AA, or
6 AA—Cysteine—2 AA—Citrulline—1 AA—Cysteine—4 AA, or
4 AA—Cysteine—2 AA—Citrulline—1 AA—Cysteine—4 AA.

8. A peptide according to claim 1-7 characterised in that the amino acids flanking the citrulline residue have a small volume and that they do not interact with the citrulline side chain.

9. A peptide according to claim 1-8 comprising the amino acid sequence
QDTIHGHPCSXXGHRCGY, or
QDTIHGHPCSSXGHRCGY, or
QDTIHGHPCSXXGHQCGY or
QDTIHGHPCSXXGHRCGQ, or
QDTIHGHPCSXXGHQCGQ, or
QDTIHGHPCSXXGCRPGY, or
HGHPCSXXGHRCGY, or
HGHPCSXXGCRPGY, or
HGHGCDXXGHRCGQ, or
HGHGCDSXGHRCGQ, or
QDTIVGWGCDSXGCRPGQ, or
VGWGCDSXGCRPGQ.

10. An antibody raised upon immunisation with a peptide according to any of the claims 1-9, with said antibody being specifically reactive with said peptide and with said antibody being preferably a monoclonal antibody.

11. An anti-idiotype antibody raised upon immunisation with an antibody according to claim 10, with said anti-idiotype antibody being specifically reactive with the antibody of claim 10, thereby mimicking a peptide according to claim 1-9, and with said antibody being preferably a monoclonal antibody.

12 A diagnostic kit for use in detecting auto-immune diseases such as rheumatoid arthritis, said kit comprising at least one peptide according to any of the claims 1-9, or an antibody according to any of the claims 10 or 11, with said peptide or antibody being possibly bound to a solid support.

13. A diagnostic kit according to claim 12, said kit comprising a range of peptides according to any of claims 1-9 or of antibodies according to any of claims 10 or 11, possibly in combination with antigens that constitute immunogenic determinants for other auto-immune diseases, wherein said peptides are attached to specific locations on a solid substrate.

14. A diagnostic kit according to claim 12 or 13, wherein said solid support is a membrane strip and said peptides are coupled to the membrane in the form of parallel lines.

15. A diagnostic kit according to claim 12 or 13 wherein certain peptides are not attached to a solid support but are provided in the binding solution to be used as competitors and/or to block other antibodies that are present in sera from patients with autoimmune disease other than rheumatoid arthritis, thereby decreasing or eliminating possible cross-reaction and/or a-specific binding.

16. Method for producing a peptide according to any of the claims 1-9, by classical chemical synthesis, wherein citrulline residues are substituted for arginine residues at certain steps during the chemical synthesis.

17. Method for producing a peptide according to claim 1-9, wherein the primary amino acid sequence is produced by classical chemical synthesis, and wherein at least one arginine residue subsequently is transformed towards a citrulline residue by contacting said peptide with a peptidylarginine deiminase.

18 An immunotoxin molecule comprising a cell recognition molecule being a peptide of any of the claims 1-9, or an antibody according to claims 10 or 11, covalently bound to a toxin molecule or active fragment thereof.

19. A peptide according to any of the claims 1-9 or an antibody according to any of the claims 10 or 11 or an immunotoxin molecule according to claim 18 or compositions thereof for use as a medicament.

20. Use of a peptide according to claims 1-9 or an antibody according to claim 10 or 11 or an immunotoxin molecule according to claim 18 or a composition thereof for the preparation of a medicament or of a diagnosticum for rheumatoid arthritis.

21. Use of a peptide according to claim 1-9 or a composition thereof for the preparation of a medicament to treat autoimmune diseases by increasing the size of antigen-immune complexes, thereby improving the clearance of the formed immune complexes.

22. Use of a peptide according to claim 1-9 or a composition thereof for the preparation of a medicament for oral or nasal administration to treat autoimmune diseases by inducing a state of systemic hyporesponsiveness or tolerance to said peptide or composition.


SEQUENCE LISTING (22 sequences)

SEQ ID NO: 1 - protein, 18 aa, Artificial Sequence
  Synthetic Peptides
XXXXXXXXCXXXXXXCXX

SEQ ID NO: 2 - protein, 15 aa, Artificial Sequence
  Synthetic Peptides
XXXXXCXXXXXXCXX

SEQ ID NO: 3 - protein, 14 aa, Artificial Sequence
  Synthetic Peptides
XXXXCXXXXXXCXX

SEQ ID NO: 4 - protein, 18 aa, Artificial Sequence
  Synthetic Peptides
XXXXXXXXCXXXXCXXXX

SEQ ID NO: 5 - protein, 16 aa, Artificial Sequence
  Synthetic Peptides
XXXXXXCXXXXCXXXX

SEQ ID NO: 6 - protein, 14 aa, Artificial Sequence
  Synthetic Peptides
XXXXCXXXXCXXXX

SEQ ID NO: 7 - protein, 18 aa, Artificial Sequence
  Synthetic Peptides
QDTIHGHPCSXXGHRCGY

SEQ ID NO: 8 - protein, 18 aa, Artificial Sequence
  Synthetic Peptides
QDTIHGHPCSSXGHRCGY

SEQ ID NO: 9 - protein, 18 aa, Artificial Sequence
  Synthetic Peptides
QDTIHGHPCSXXGHQCGY

SEQ ID NO: 10 - protein, 18 aa, Artificial Sequence
  Synthetic Peptides
QDTIHGHPCSXXGHRCGQ

SEQ ID NO: 11 - protein, 18 aa, Artificial Sequence
  Synthetic Peptides
QDTIHGHPCSXXGHQCGQ

SEQ ID NO: 12 - protein, 18 aa, Artificial Sequence
  Synthetic Peptides
QDTIHGHPCSXXGCRPGY

SEQ ID NO: 13 - protein, 14 aa, Artificial Sequence
  Synthetic Peptides
HGHPCSXXGHRCGY

SEQ ID NO: 14 - protein, 14 aa, Artificial Sequence
  Synthetic Peptides
HGHPCSXXGCRPGY

SEQ ID NO: 15 - protein, 14 aa, Artificial Sequence
  Synthetic Peptides
HGHGCDXXGHRCGQ

SEQ ID NO: 16 - protein, 14 aa, Artificial Sequence
  Synthetic Peptides
HGHGCDSXGHRCGQ

SEQ ID NO: 17 - protein, 18 aa, Artificial Sequence
  Synthetic Peptides
QDTIVGWGCDSXGCRPGQ

SEQ ID NO: 18 - protein, 14 aa, Artificial Sequence
  Synthetic Peptides
VGWGCDSXGCRPGQ

SEQ ID NO: 19 - protein, 14 aa, Artificial Sequence
  Synthetic Peptides
TGSSTGGXQGSHHE

SEQ ID NO: 20 - protein, 14 aa, Artificial Sequence
  Synthetic Peptides
ESSRDGSXHPRSHD

SEQ ID NO: 21 - protein, 14 aa, Artificial Sequence
  Synthetic Peptides
EQSADSSXHSGSGH

SEQ ID NO: 22 - protein, 22 aa, Artificial Sequence
  Synthetic Peptides
SHQCHQESTXGRSRGRCGRSGS